(19) United States
(12) Patent Application Publication
(10) Pub. No.: US 2008/0214894 A1
(43) Pub. Date: Sep. 4, 2008

(54) Robotic endoscopy actuator

(76) Inventor: Matthias Wedel, Nurnberg (DE)

Correspondence Address: BRINKS HOFER GILSON & LIONE, P.O. BOX 10395, CHICAGO, IL, 60610, US

(21) Appl. No.: 11/788,177

(22) Filed: Apr. 18, 2007

(30) Foreign Application Priority Data
Apr. 26, 2006 (DE) DE 102006019419.5

(51) Int. Cl. A61B 1/00

(52) U.S. Cl. 600/118

(57) ABSTRACT

A robotic endoscopy actuator is provided. The robotic endoscopy actuator includes a function means; and an energy absorption element that is operable to absorb energy from an electromagnetic field. The energy absorption element includes a heat element. The function means is operable to fulfill a function using heat energy.

The present patent document claims the benefit of the filing date of DE 10 2006 019 419.5, filed Apr. 26, 2006, which is hereby incorporated by reference.

### BACKGROUND

The present embodiments relate to a robotic endoscopy actuator, for example, an endorobot actuator.

Conventional endoscopy uses an elongated endoscopic device for insertion into the organ or vessel for diagnosis of diseases. DE 10 2005 006 877 A1 discloses a capsule endoscopy that may also be used for the diagnosis of diseases, such as diseases of the gastrointestinal tract. During endoscopy, a mobile part of an endorobot is introduced into the organ or vessel and controlled by a stationary part of the endorobot arranged outside the patient. During an examination of the gastrointestinal tract, the mobile part is swallowed by the patient. The mobile part moves through the body, propelled by peristalsis. Inside the patient, the mobile part of the endorobot executes certain functions, for example, records a number of images for diagnosing the organ or vessel, takes samples, or clamps wounds. In order to control an intended movement of the mobile part, a magnetic field is applied externally. The magnetic field also supplies a function element of the mobile part with current for executing the desired function.

Generally, mechanical parts, such as a motor or a gear unit, demand high input and as a result are prone to faults. Such actuators are large or have only limited actuating forces. A power supply via a cable is difficult to use with an actuator of an endorobot.

### SUMMARY

The present embodiments may obviate one or more of the limitations or drawbacks inherent in the related art. For example, in one embodiment, an endoscopy device includes a small, simple or non-fault-prone mobile part of an endorobot.

In one embodiment, an energy absorption element has a heat element and a function unit is able to fulfill a function through heat energy. A useful movement can be driven by heat, and a simple, very small and robust design of the actuator may be achieved.

An endorobot is a robot that can operate at generally inaccessible points inside a body, such as a human body, without tissue-destroying intervention. The electromagnetic field is an alternating field. The energy absorption element may be identical with the heat element. The heat element has a substance that absorbs energy from the electromagnetic field, such as an alternating field. The substance may be for example, ferrite material, resistance wire, or iron powder. Other suitable substances may be used, such as active powder or granulated material, a coil or another solid or a liquid. Remagnetization losses in iron or ferritic material or else ohmic losses may be utilized.

In one embodiment, the heat element may absorb energy direct from the electromagnetic field. The energy may be made directly available as working energy. The heat element converts energy from the electromagnetic field directly into heat. Consequently, conversion of the energy from the electromagnetic field into, for example, electrical energy, is not required.

In one embodiment, a movement is generated. The heat element applies the force or energy needed for the movement. A large mechanical force may be generated in a simple manner and with a high degree of efficiency.

In one embodiment, the actuator may be maintained in a robust condition. The function unit, in conjunction with a deformation produced by heating of the heat element, may execute a working movement. The function unit may be deformed. The function unit may include, for example, a piece of memory metal, which in a cold state stays in a first shape condition and when heated sufficiently passes into a second shape condition. The function unit may include, for example, a bimetal, which is deformed upon input of heat.

In one embodiment, the heat element may be deformed upon heating and cooling. The deformation movement may be transferred to the function unit, which executes the working movement. The heat element may be deformed through heating, as a result of which a simple design of the actuator is possible. The heat element may include a deformable medium held in a wall. The wall may be deformed when the heat element is deformed. The wall may enclose a volume. The deformable medium may remain enclosed by the wall when the volume is changed in shape and/or size by the heating. The wall may be expandable.

In one embodiment, the heat element may include a fluid that heats up, by virtue of which a change in the heating of the heat element may be achieved. The fluid is a liquid, a gas or a gel-like substance. If the fluid is a gas, then through heat input, a continuous change in volume of the fluid can be achieved. A steady movement of the function unit can be achieved. If the fluid is fashioned as a liquid or gel, the fluid may, through heat input, be evaporated so that a large volume change and thus a large functional movement may be achieved.

The fluid deforms the heat element by a phase transition. The fluid has a boiling point, which lies only a few degrees above human body temperature, for example, between about 43° C. and 55° C. The fluid has a low heat capacity in the phase transition so that the heat input may be kept low. In a phase transition into the liquid or gel-like phase, the fluid emits only limited heat. In one embodiment, the fluid may include a mixture of gas and liquid, the quantity of liquid may determine a final size reached after full evaporation and the gas may determine initial size of the heat element existing prior to evaporation.

In one embodiment, a function unit has an inner cavity with an outlet. The heat element presses, by a change in size, a substance out of the outlet. For example, when the actuator reaches a location in the body intended for a substance dose, the heat element may be heated and the substance pressed out of the inner cavity.

In one embodiment, the heat element is prepared for the absorption of electromagnetic radiation from a predefined first absorption frequency band. The heat element may not substantially absorb or may only absorb in a limited way electromagnetic radiation from an adjacent second frequency band. Interference with control through the unwanted irradiation of electromagnetic radiation may be counteracted. The absorption frequency band is narrow.

In one embodiment, the actuator has a plurality of heat elements that can be controlled separately. A function may be executed using a plurality of subfunctions. A large variety of functions may be executed using the subfunctions. For example, a complicated movement sequence may be composed of a series of individual movements.

In one embodiment, the actuator may have a plurality of heat elements that absorb electromagnetic radiation from different absorption frequency bands. Depending on the frequency of an inducing electromagnetic field, a defined heat element may be controlled or a plurality of heat elements may be controlled simultaneously. Each heat element corresponds to one of the absorption frequency bands, which the heat element absorbs and leaves the other frequency bands unabsorbed.

In one embodiment, an endorobot includes an actuator and a control unit that controls the actuator. The actuator may be mechanically separated from the control unit. The actuator may be used inside a human body. The control unit may remain outside the human body. The endorobot may also include a transmitter that radiates an electromagnetic field. The control unit may be mechanically rigidly connected (fixed) to the transmitter. The control unit may be coupled mechanically (directly or indirectly) to the actuator. For example, the actuator may include the control unit. The control unit may transmit (communicate) transmit commands from inside the body to the transmitter arranged outside the body.

In one embodiment, the control unit controls a plurality of heat elements. The heat elements have a frequency assigned to the respective heat element. The frequencies differ from one another. A plurality of heat elements may be controlled independently and a variety of functions achieved. The frequencies may be frequency bands with a predetermined bandwidth.

Control of the actuator may be achieved by a sensor that determines the size of the heat element. An operating status of the heat element may be determined, for example whether the heat element is currently large and executing an operating function or whether it is small and the operating function, for example, a movement, has been retracted. Depending on the current operating status of the heat element, a further operation may be initiated by the control unit. The size may be determined by ultrasound or by transillumination, for example, by X-ray radiation. A size of a volume of gas in a surrounding liquid may be determined by the sharp contrast between liquid and gas. A size change may be monitored by the control unit. A precise determination of a current operating status may be made based on the size change.

Control of the actuator may also be based on a sensor that determines an energy absorption of the heat element. Depending on the energy absorption, it can be concluded how far the heat element has heated up and a current operating status can be determined from this. The energy absorption may be determined from a damping of the electromagnetic field.

In one embodiment, a sensor may determine a shift of an absorption frequency band through a movement of the heat element or of the function means. The actuator may change absorption frequency bands when there is a change in the shape of the heat element or of the function means. A shape of the heat element may be determined by measuring the damping of the electromagnetic field at selected frequencies.

In one embodiment, the inductance of the oscillating circuit of transmitter and actuator may be changed. An operating status may be determined based on the change of inductance. The energy absorption or the damping of the heat element may be measured purely qualitatively, for example, only as a relative change in an energy absorption, or quantitatively.

In one embodiment, the endorobot comprises a plurality of sensors for the independent monitoring of a plurality of heat elements. A complicated operating sequence may be monitored reliably using the plurality of sensors.

### BRIEF DESCRIPTION OF THE DRAWINGS

FIG. 1 shows a patient having received one embodiment of an endorobot,

FIG. 2 shows an actuator of the endorobot depicted in FIG. 1,

FIG. 3 shows four further actuators of an endorobot,

FIG. 4 shows one embodiment of an actuator in open and closed position,

FIG. 5 shows one embodiment of an actuator in passive and active position,

FIG. 6 shows one embodiment of a tripod with three actuators,

FIG. 7 shows one embodiment of an actuator for expanding in passive and active position,

FIG. 8 shows one embodiment of an actuator for holding in a vessel, in passive and active position,

FIG. 9 shows one embodiment of an actuator that expels a fluid in passive and active position,

FIG. 10 shows one embodiment of an actuator for controlled moving,

FIG. 11 shows one embodiment of an actuator as depicted in FIG. 10 in triple active status and

FIG. 12 shows one embodiment of a movement sequence in a vessel of the actuator from FIGS. 10 and 11, with a control model.

### DETAILED DESCRIPTION

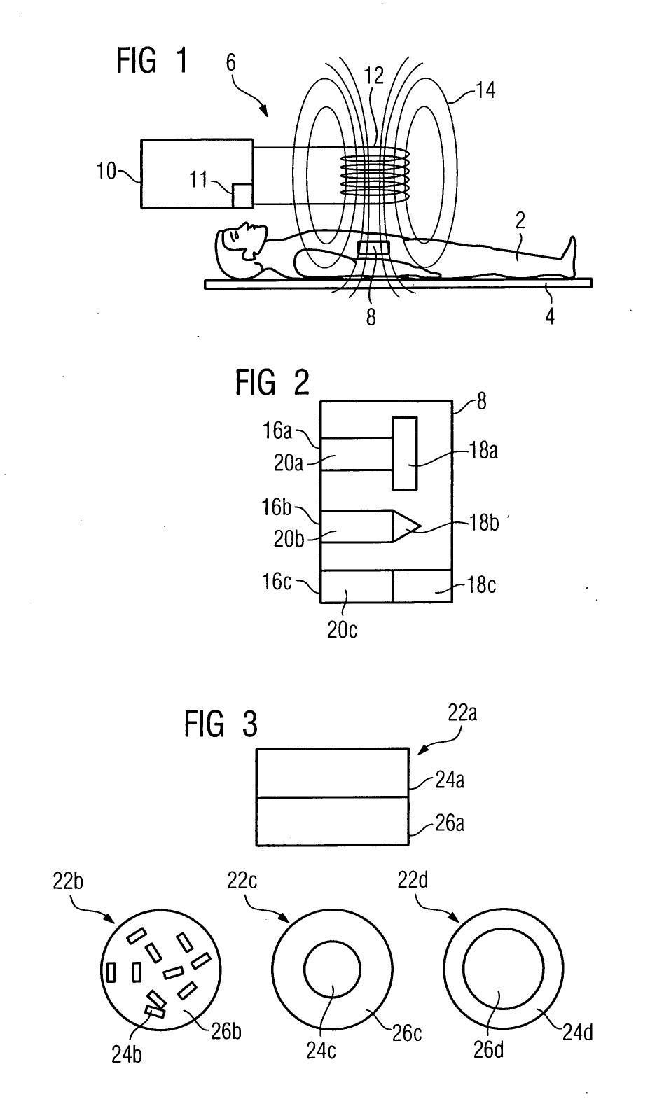

FIG. 1 shows a patient 2 on a bed 4 with an endorobot 6, which has an actuator 8, shown only schematically in FIG. 1, a control unit 10 with a sensor 11 and a transmission wire 12. The transmission wire 12 includes a transmit and receive coil, which generates an alternating electromagnetic field 14 and receives the alternating field 14. The sensor 11 or control unit 10 measures the alternating field 14. The control unit 10 may excite the alternating field 14 with one or more adjustable fixed or variable frequencies and may evaluate the receive signal received from the coil.

FIG. 2 shows the actuator 8 of the endorobot as depicted in FIG. 1. The actuator 8 includes three energy absorption elements in the form of heat elements 16a-c. The heat elements 16a-c may be connected to a function unit 18a-c. The first heat element 16a may absorb electromagnetic radiation 14, for example, radio radiation, through induction from a first absorption frequency band. The first absorption frequency band corresponds to material 20a of the heat element 16a, for example, ferrite material, in such a way that the material 20a can readily absorb the electromagnetic radiation 14 and can readily convert it into heat through remagnetization losses. The heat elements 16b and 16c may be embodied similar to the heat element 16a. The heat elements 16b and 16c may comprise a slightly different material 20b, 20c, corresponding to a second or third absorption frequency band. The three absorption frequency bands are slightly different in their frequency position and do not overlap. Each heat element 16a-c leaves electromagnetic radiation from one of the adjacent frequency bands essentially unabsorbed. The three heat elements 16a-c may be controlled by the control unit 10 separately through three different excitation frequencies. The three function units 18a-c are fashioned fulfill their own function.

FIG. 3 shows four different actuators 22a-d that include heat elements 24a-d and function unit 26a-d. In the actuator 22a, the heat element 24a and the function unit 26a are arranged in layers on top of one another. In the actuator 22b, the heat element 24b includes many small elements in the function unit 26b. Actuator 22c includes a heat element 24c that is arranged inside the function unit 26c. Actuator 22d includes a heat element 24d that is arranged outside the function unit 26d. The position of the heat elements 24a-d in relation to their function units 26a-d is determined by the function to be fulfilled by the function units 26a-d.

The actuators 8, 22a-d may cool the heat elements 16a-c, 24a-d. The heat elements 16a-c, 24a-d may be arranged on the outside in the actuator 8, 22a, 22d and/or have a heat transfer unit that transfers heat from the heat element 16a-c, 24b, 24c to outside the actuator 8, 22b, 22c. The heat transfer unit may include a function unit 26b, 26c, which is provided for the transfer of heat. The thermal connection of the heat elements 16a-c, 24a-d to the surroundings of the actuator 8, 22a-d enables the heat elements 16a-c, 24a-d to cool rapidly after heating. The respective function units 18a-c, 26a-d may return rapidly to its initial status, for example, its starting position.

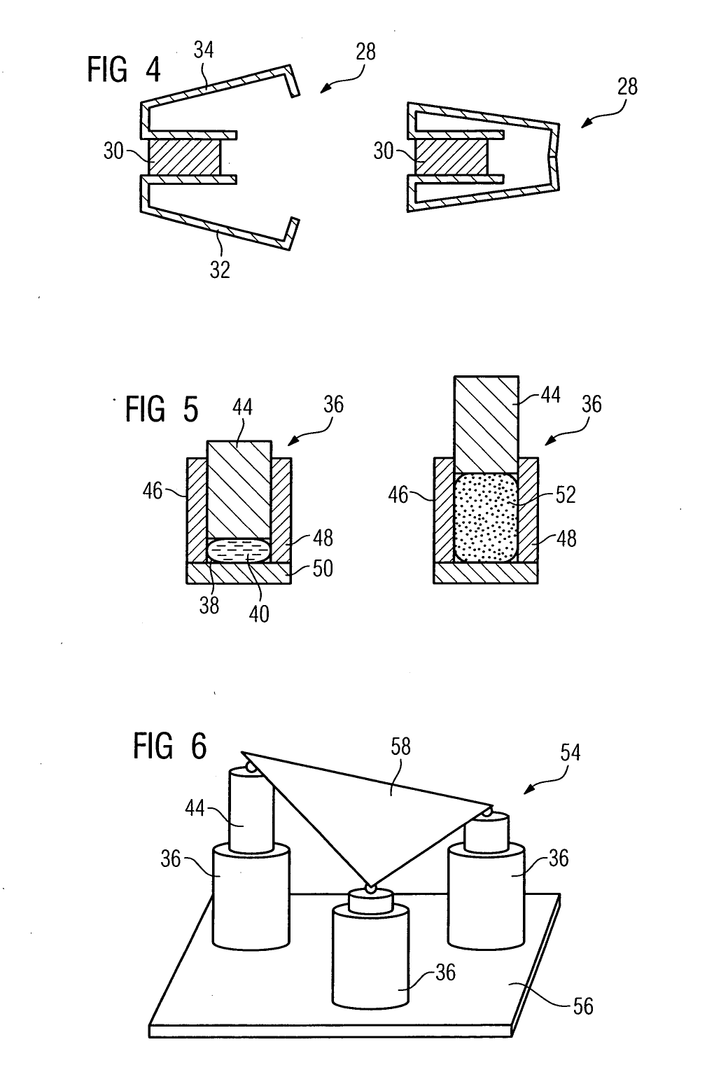

FIGS. 4 to 12 show additional embodiments of actuators 28, 36, 60, 72, 84, 98. The mode of operation is analogous to that of the above-described actuators 8, 22a-d.

FIG. 4 shows one embodiment of an actuator 28 that includes a heat element 30 and a function unit 32 with two gripping arms 34, which are shown on the left-hand side of FIG. 4 in the open position and on the right-hand side of FIG. 4 in the closed position. One or both of the two gripping arms 34, which rest in the open position when a heat element is cold, include memory metal. When the heat element 30 is heated, heat is transferred from the heat element 30 to the gripping arms 34. At a predetermined temperature, the gripping arms 34 move into the closed position and remain there for as long as their temperature lies above the predetermined temperature. The gripping arms 34 may be used to grip (hold) a piece of tissue. The gripping arms 34 may be used to separate the gripped tissue from other tissue.

In one embodiment, as shown in FIG. 5, the actuator 36 includes a heat element 38 and a function unit 44. The heat element 38 may include an expandable container 42 filled with liquid 40. The function unit 44 may include a die. The heat element 38 and function unit 44 may be disposed in a housing 46. The housing 46 may include a wall 48 and a floor 50. When the heat element 38 is heated, the liquid 40 is heated through the direct absorption of electromagnetic radiation or through radiation-absorbing particles, for example, ferrite particles. The liquid 40 may include the radiation-absorbing particles. The boiling point of the liquid 40 may be around 45° C. The heat capacity of the liquid 40 may be low. The liquid 40 may boil even when a low amount of heat is transferred to the liquid 40. The container 42 may fill with gas 52 and expand. The die executes a working movement by being forced out of the housing 46. When cooled, the die travels back into the housing 46 again. Alternatively, the floor 50 may include a heat element that transfers its heat to the liquid 40.

As shown in FIG. 6, a tripod 54 includes three actuators 36. The tripod includes a base plate 56 and a working plate 58. The heat elements 44 of the actuators 36 are set to different absorption frequency bands. The actuators 36 may be controlled independently of each other. The working plate 58 may be moved in three axes of freedom, for example, may be swiveled two-dimensionally and raised and lowered in the direction of displacement of the function units 44. A tripod 54 may be used, for example, for moving a camera.

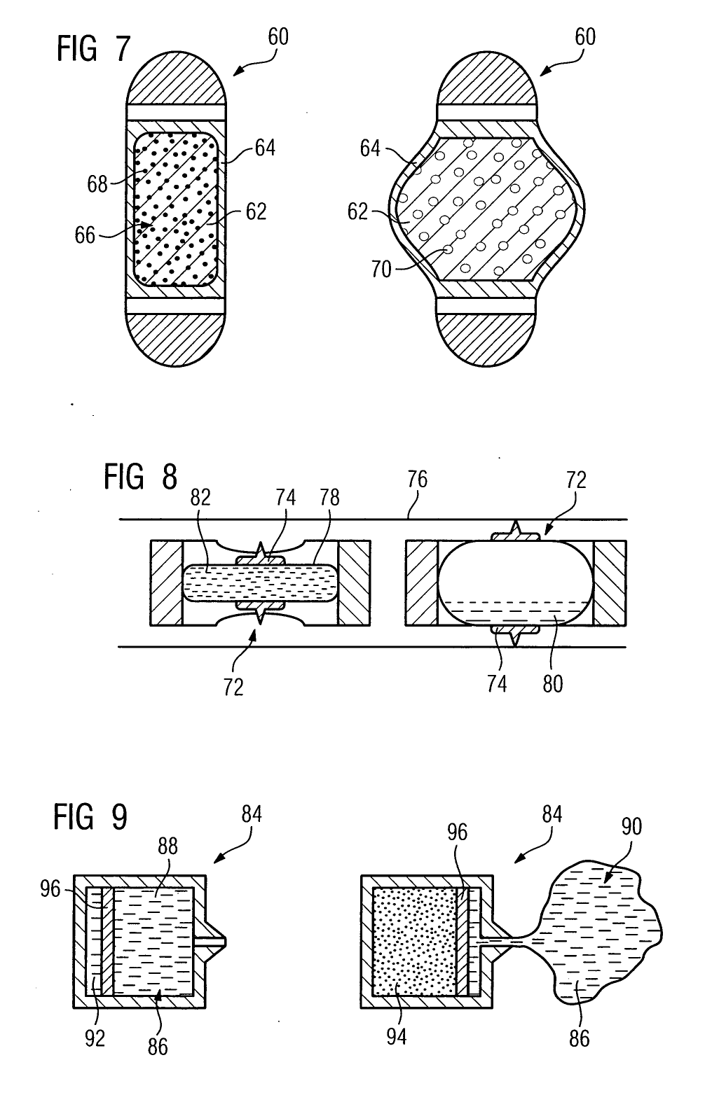

In one embodiment, as shown in FIG. 7, an actuator 60 includes a heat element 62 and a function unit 64 having an outer skin. The heat element 62 includes an elastic material 66, for example, a gel or an elastomer. The elastic material 66 absorbs energy from an alternating electromagnetic field either of its own accord or with the aid of embedded particles. The elastic material 66 may include liquid bubbles 68, the liquid of which evaporates when heated sufficiently and gas bubbles 70 form as a result to cause an expansion of the outer skin. As shown in the right side of FIG. 7, a vessel may be expanded, for example, by using gas bubbles 70.

In one embodiment, as shown in a sectional view of FIG. 8, an actuator 72 includes a function unit 74 for holding in a vessel 76. The heat element 78 of the actuator 72 includes a mixture of an absorption liquid 80 that absorbs energy from an alternating electromagnetic field and a liquid 72 that evaporates. The function unit 74 like the heat element 78 is elastic and may be directed (connected) around the heat element 78. A plurality of separate holding elements may form the function unit 74.

As shown in FIG. 9, the actuator 84 may expel a medically active liquid 86 from an inner cavity 88 into the surroundings 90 of the actuator 84. The actuator 84 may include a liquid 92 that serves as a heat element. When heated, the liquid 92 evaporates to form a gas 94. The gas 94 displaces a die 96, which forces the liquid 86 out of the inner cavity 88.

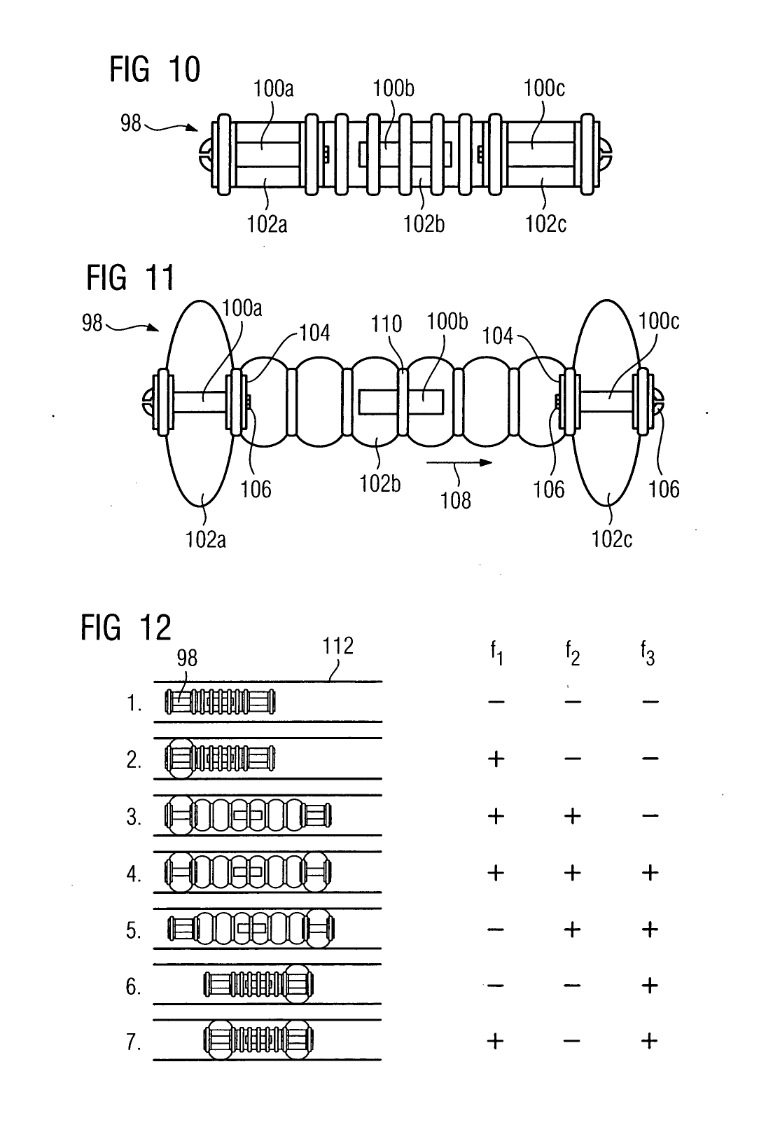

As shown in FIGS. 10 and 11, an actuator 98 is used for a targeted movement. The actuator 98 includes three separately controllable heat elements 100a-c. The heat elements 100a-c lie in an evaporable medium that is distributed between three chambers 102a-c. The chambers 102a-c are separated from one another in a gastight manner by two seals 104. The chambers 102a-c may be expanded separately by the evaporable medium. The two outer chambers 102a, 102c are held constant in their expansion in an axial direction by two holders 106, for example, a screw directed through the heat element 100a, 100c. The central chamber 102b is limited in its expansion perpendicular to the axial direction by retaining rings 110. FIG. 10 shows the actuator 98 in tension-relieved status, for example, with cool heat elements 100a-c. FIG. 11 shows the actuator 98 with evaporated medium and maximally expanded chambers 102a-c.

FIG. 12 shows seven acts of movement of the actuator 98 through a vessel 112. Shown in tabular form on the right-hand side of FIG. 12 are the frequencies f₁, f₂ and f₃ with which the transmission medium 12 radiates the alternating electromagnetic field. The heat element 100a absorbs radiation with the frequency f₁, the heat element 100b absorbs radiation with the frequency f₂, and the heat element 100c absorbs radiation with the frequency f₃. The heat elements 100a-c leave radiation with the other two frequencies f₁, f₂ or f₃ essentially unabsorbed.

In a first act, no alternating electromagnetic field radiates from the transmission medium. Consequently, all three heat elements 100a-c are cool. The medium is relieved of tension everywhere and the chambers 102a-c are not expanded. In the second to the fourth acts, the transmission medium 12 radiates an alternating electromagnetic field with the frequency f₁, then with f₂ and f₃, and with all three frequencies f₁, f₂ and f₃. Initially only the first heat element 102a, then two heat elements 102a, 102b, and then all three heat elements 102a-c are heated. The actuator 98 in the vessel 112 is tensioned, expanded and then doubly tensioned.

In the fifth act, through switching off of the first frequency f₁, the heat element 100a emits its heat rapidly to the surroundings and cools down rapidly. The chamber 102a is relieved of tension. In the sixth act, the chamber 102a may be pulled by relieving tension of the second chamber 102b to the third chamber 102c. In the seventh act, the chamber 102a is again expanded to double the tension in the vessel 112. The movement process recommences with a fresh cycle from the second to the seventh acts. The cycle may be repeated for targeted movement through the vessel 112. The movement may be controlled by the control unit 10. Movement through a curved vessel is also possible without problems. The control unit controls the heat elements 100a-c using the frequency f₁, f₂, f₃ respectively assigned to the respective heat element 100a-c.

In one embodiment, the control unit 10 monitors behavior of the heat elements 16a-c, 24a-d, 30, 38, 62, 78, 100a-c with the aid of the sensor 11 and/or the coil. The sensor 11 serves to determine the size of the heat element 16a-c, 24a-d, 30, 38, 62, 78, 100a-c or volume of gas by ultrasound or X-ray radiation and/or to determine an energy absorption of the heat element 16a-c, 24a-d, 30, 38, 62, 78, 100a-c via damping of the alternating field. The control unit 10 may vary a frequency of the alternating field and to determine an absorption depending on the frequency. This produces an absorption displacement from which the control unit 10 determines with the aid of previously determined empirical data a movement or size status of the heat elements 16a-c, 24a-d, 30, 38, 62, 78, 100a-c. The sensor 11 may include a plurality of sensor elements. The plurality of sensor elements may monitor independently a plurality of heat elements 16a-c, 24a-d, 30, 38, 62, 78, 100a-c.

Various embodiments described herein can be used alone or in combination with one another. The forgoing detailed description has described only a few of the many possible implementations of the present invention. For this reason, this detailed description is intended by way of illustration, and not by way of limitation. It is only the following claims, including all equivalents that are intended to define the scope of this invention.

1. A robotic endoscopy actuator comprising:
a function unit operable using heat energy; and
an energy absorption element operable to absorb energy from an electromagnetic field,
wherein the energy absorption element comprises a heat element operable to provide the heat energy to the function unit.

2. The robotic endoscopy actuator as claimed in claim 1, wherein the heat element is operable to directly absorb the energy from the electromagnetic field.

3. The robotic endoscopy actuator as claimed in claim 2, wherein the function unit is operable to generate a movement and the heat element is operable to apply a force needed for the movement.

4. The robotic endoscopy actuator as claimed in claim 3, wherein the function unit deforms by a heating of the heat element.

5. The robotic endoscopy actuator as claimed in claim 1, wherein the heat element is operable to be deformed through heating.

6. The robotic endoscopy actuator as claimed in claim 1, wherein the heat element comprises a fluid that is operable to be heated.

7. The robotic endoscopy actuator as claimed in claim 6,
wherein the fluid is provided for a deformation of the heat element through a phase transition.

8. The robotic endoscopy actuator as claimed in claim 1, wherein the function unit comprises an inner cavity with an outlet, the heat element being operable to force a substance from the outlet by a size change.

9. The robotic endoscopy actuator as claimed in claim 1, wherein the heat element is operable to absorb electromagnetic radiation from a first absorption frequency band.

10. The robotic endoscopy actuator as claimed in claim 1, comprising a plurality of heat elements that can be controlled separately.

11. The robotic endoscopy actuator as claimed in claim 1, comprising a plurality of heat elements that are operable to absorb electromagnetic radiation from absorption frequency bands.

12. An endorobot comprising:
an actuator that includes a function unit and an energy absorption element operable to absorb energy from an electromagnetic field; and
a control unit that is operable to control the actuator.

13. The endorobot as claimed in claim 12, wherein the control unit is operable to control a plurality of heat elements, each of the plurality of heat elements has a frequency assigned to the respective heat element, wherein the frequencies differ from one another.

14. The endorobot as claimed in claim 12, comprising a sensor for determining a size of the heat element.

15. The endorobot as claimed in claim 12, wherein a sensor is operable to determine an energy absorption of the heat element.

16. The endorobot as claimed in claim 12, wherein a sensor is operable to determine a shift of an absorption frequency band based on a movement of the heat element.

17. The endorobot as claimed in claim 12, comprising a plurality of sensors that are operable to monitor a respective plurality of heat elements.

18. The robotic endoscopy actuator as claimed in claim 9, wherein the heat element is operable to leave electromagnetic radiation from a second frequency band substantially unabsorbed.
